(19) United States
(12) Patent Application Publication
(10) Pub. No.: US 2019/0392939 A1
(43) Pub. Date: Dec. 26, 2019

(54) SYSTEMS AND METHODS FOR MANAGEMENT AND SCHEDULING OF DIFFERENTIAL AIR PRESSURE AND OTHER UNWEIGHTED OR ASSISTED TREATMENT SYSTEMS

(71) Applicant: AlterG, Inc., Fremont, CA (US)

(72) Inventors: Steven L. BASTA, Menlo Park, CA (US); Michael E. DUFFY, Sebastopol, CA (US); Clifford T. JUE, Santa Cruz, CA (US)

(21) Appl. No.: 16/254,503

(22) Filed: Jan. 22, 2019

Related U.S. Application Data
(63) Continuation of application No. 14/769,113, filed on Aug. 20, 2015, now abandoned.
(60) Provisional application No. 61/785,402, filed on Mar. 14, 2013.

(51) Int. Cl. G16H 20/30

(52) U.S. Cl. CPC G16H 20/30

(57) ABSTRACT

Systems and methods for management and scheduling of differential air pressure (DAP) and other unweighted or assisted treatment systems are provided. The methods and systems can include generation of a suggested workout based on matching user data to data of other users sharing similar characteristics using an aggregate database of user information and related workout information. The methods and systems can include matching a user to an available and/or appropriate DAP system. Also provided are methods and systems including performing a workout and uploading performance information to an aggregate database of user information.

### CROSS REFERENCE TO RELATED APPLICATIONS

This application is a continuation of U.S. patent application Ser. No. 14/769,113, filed Aug. 20, 2015, titled “SYSTEMS AND METHODS FOR MANAGEMENT AND SCHEDULING OF DIFFERENTIAL AIR PRESSURE AND OTHER UNWEIGHTED OR ASSISTED TREATMENT SYSTEMS,” now U.S. Patent Application Publication No. 2015/0379239, which is a national phase application under 35 USC 371 of International Patent Application No. PCT/US2014/028032, filed Mar. 14, 2014, titled “SYSTEMS AND METHODS FOR MANAGEMENT AND SCHEDULING OF DIFFERENTIAL AIR PRESSURE AND OTHER UNWEIGHTED OR ASSISTED TREATMENT SYSTEMS” now International Publication No. WO 2014/152862, which claims the benefit of U.S. Provisional Application No. 61/785,402, filed Mar. 14, 2013. This application may be related to any of the following patent applications, each of which is herein incorporated by reference in its entirety: U.S. Pat. No. 7,591,795 issued on Sep. 22, 2009; U.S. application Ser. No. 12/236,459 filed on Sep. 23, 2008; U.S. application Ser. No. 12/236,465 filed on Sep. 23, 2008; U.S. application Ser. No. 12/236,468 filed on Sep. 23, 2008; International Application No. PCT/US2006/038591 filed on Sep. 28, 2006; U.S. Provisional Application No. 60/999,102 filed on Oct. 15, 2007; U.S. Provisional Application No. 60/999,101 filed on Oct. 15, 2007; U.S. Provisional Application No. 60/999,061 filed on Oct. 15, 2007; U.S. Provisional Application No. 60/999,060 filed on Oct. 15, 2007; U.S. application Ser. No. 12/761,316 filed on Apr. 15, 2010; U.S. application Ser. No. 12/761,312 filed on Apr. 15, 2010; International Application No. PCT/US2008/011832 filed on Oct. 15, 2008; International Application No. PCT/US2008/011807 filed on Oct. 15, 2008; U.S. Provisional Application No. 61/178,901 filed on May 15, 2009; U.S. application Ser. No. 12/778,747 filed on May 12, 2010; International Application No. PCT/US2010/034518 filed on May 12, 2010; U.S. Design application No. 29/337,097 filed on May 14, 2009; U.S. Provisional Application No. 61/454,432 filed on Mar. 18, 2011; U.S. application Ser. No. 13/423,124 filed on Mar. 16, 2012; International Application No. PCT/US12/29554 filed on Mar. 16, 2012; U.S. Pat. No. 5,133,339 issued on Jul. 28, 1992; U.S. Provisional Application No. 61/651,415 filed on May 24, 2012; U.S. Provisional Application No. 61/785,317, filed Mar. 14, 2013, titled “METHOD OF GAIT EVALUATION AND TRAINING WITH DIFFERENTIAL PRESSURE SYSTEM,” filed herewith; U.S. Provisional Application No. 61/784,387, filed Mar. 14, 2013, titled “SUPPORT FRAME AND RELATED UNWEIGHTING SYSTEM,” filed herewith; U.S. Provisional Application No. 61/772,964 filed on Mar. 5, 2013; U.S. Provisional Application No. 61/773,019 filed on Mar. 5, 2013; U.S. Provisional Application No. 61/773,037 filed on Mar. 5, 2013; U.S. Provisional Application No. 61/773,048 filed on Mar. 5, 2013; U.S. Provisional Application No. 61/784,664, filed Mar. 14, 2013 titled “UNWEIGHTING GARMENTS,” filed herewith and U.S. Provisional Application No. 61/784,510, filed Mar. 14, 2013 titled “CANTILEVERED UNWEIGHTING SYSTEMS,” filed herewith, each of which are incorporated by reference its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

Aspects of this invention generally relate to systems and methods for scheduling and managing treatment provided by assisted training systems such as differential air pressure systems as well as other personal assistance or unweighing systems. More particularly, embodiments of the invention relate to management of treatment resources and schedules such that patients in need of therapeutic treatment can access available appropriate treatments from unweighting and assistive training systems regardless of type of treatment or location and timing of treatments. Further embodiments of this invention relate to multimodality therapy involving unweighting, personalized assistive, and various types of DAP systems and other forms of rehabilitation therapy, and relate to the scheduling and integration of multiple modes of therapy such as alternating time on a DAP to improve walking with flexibility, stretching or strength training protocols. Such multiple modes of therapy can integrate input and data captured from the unweighted therapy or assisted therapy session, patient-provided information, information from the medical records system of the therapy center, or information captured from other therapeutic rehab equipment such as bicycles, or strength testing equipment.

### BACKGROUND

Methods of counteracting gravitational forces on the human body have been devised for therapeutic applications as well as physical training. Rehabilitation from orthopedic injuries or neurological conditions, particularly as they pertain to the lower portion of the body, often benefits from precision unweighting (i.e. partial weight bearing) therapy, which allows the patient to more easily rehab or train their lower limbs. One way to counteract the effects of gravity is to suspend a person using a body harness to reduce ground impact forces. However, harness systems may cause pressure points that may lead to discomfort which restricts the amount and type of rehab or training that is possible for a given patient. Another approach to counteract the effects of gravity is to submerge a portion of a user's body into water and let buoyancy provided by the water offset gravity. However, immersion in water is problematic for patients with open wounds and induces significant lateral resistance to movement which can inhibit the recovery process, particularly with regards to restoring normal gait and the correct muscle activation patterns required for normal gait.

Differential Air Pressure (DAP) systems (such as those described in detail in U.S. Pat. No. 7,591,795 issued on Sep. 22, 2009, U.S. application Ser. No. 12/236,459 filed on Sep. 23, 2008, U.S. application Ser. No. 12/236,465 filed on Sep. 23, 2008, U.S. application Ser. No. 12/236,468 filed on Sep. 23, 2008, International Application No. PCT/US2006/038591 filed on Sep. 28, 2006, U.S. Application No. 60/999,102 filed on Oct. 15, 2007, U.S. Application No. 60/999,101 filed on Oct. 15, 2007, U.S. Application No. 60/999,061 filed on Oct. 15, 2007, U.S. Application No. 60/999,060 filed on Oct. 15, 2007, U.S. application Ser. No. 12/761,316 filed on Apr. 15, 2010, U.S. application Ser. No. 12/761,312 filed on Apr. 15, 2010, International Application No. PCT/US2008/011832 filed on Oct. 15, 2008, International Application No. PCT/US2008/011807 filed on Oct. 15, 2008, U.S. Application No. 61/178,901 filed on May 15, 2009, U.S. application Ser. No. 12/778,747 filed on May 12, 2010, PCT/US2010/034518 filed on May 12, 2010, U.S. application Ser. No. 29/337,097 filed on May 14, 2009, International Application No. 61/454,432 filed on Mar. 18, 2011, U.S. application Ser. No. 13/423,124 filed on Mar. 16, 2012, International Application No. PCT/US12/29554 filed on Mar. 16, 2012 and U.S. Pat. No. 5,133,339 issued on Jul. 28, 1992) have been developed to use air pressure in, for example, a sealed chamber surrounding the user's lower body to simulate a low gravity effect and support a patient at his center of gravity without the discomfort of harness systems or the inconvenience of water-based therapies. Scheduling of therapy to take advantage of DAP's unique benefits poses several unique challenges. First, different users have different physical accommodation needs. For example, a wheelchair bound patient in need of using a DAP system to receive rehabilitative treatment will require a DAP system having an access assist device allowing the patient to access the chamber. DAP systems typically include a chamber into which a user must step into or climb into. For a wheelchair bound patient, the appropriate DAP system would include an access assist feature to help maneuver the user into the DAP system for treatment. As can be appreciated, DAP system can come in a variety of shapes, sizes, and features. As such, not every DAP system is appropriate for use with every patient class. For example, range of mobility can be a limiting factor for whether a particular user can use a particular DAP system. A severely mobility impaired user such as a stroke patient may need a DAP system with an access assist feature, while an injured athlete recovering from a sprained ankle may not need the same level of assistance to use a DAP system. Similarly, different DAP systems may be designed for different degrees of unweighting and for different user parameters—a system optimum for a stroke patient to relearn walking for example may provide 80% unweighting and would only need to accommodate moderate walking speeds with optimization for low speed function of the treadmill; while a system appropriate for an injured athlete might not need the same degree of unweighting, but the treadmill would need to be designed to permit higher speeds for running. Accordingly, there is a need for a treatment management and scheduling method and system that considers a user's needs and pairs the user with the appropriate DAP system.

In addition, therapy often consists of a series of treatment sessions, with each session requiring different DAP system setup parameters. In contrast to scheduling systems that only arrange generic, discrete events such as taxi rides or dining times, the need exists for a bespoke scheduling system that accommodates scheduling of a series of changing events.

In addition, finding the appropriate DAP system also requires locating an available system at a geographically acceptable distance from the user. As can be appreciated, a user's therapeutic DAP treatment may require several sessions. For example, a total knee replacement patient will need multiple sessions of physical therapy with DAP in order to regain usage of the affected leg. However, the number of DAP systems is finite and often times facilities only have one machine. This is a relatively low number of DAP systems compared to the number of patients that may need to use a DAP system at any given time. Therefore, there is a need for a system and method of tracking the availability of DAP systems at a geographically acceptable distance from a user. In some cases, DAP therapy may be one of several modes of rehabilitation therapy applied, which raises additional requirements regarding the breadth of available equipment in a rehabilitation facility. If therapy involving both DAP and other modes of rehabilitation in a single session or in multiple sessions is required, identifying facilities with the appropriate equipment and availability for the multiple modes of treatment may be challenging or inefficient without a method for automating the scheduling of such therapy. Further, automated scheduling may improve efficiency to such a degree that improved compliance by patients is achieved—since a patient who has difficulty scheduling treatment sessions or has to make multiple visits to one or more clinics to use multiple pieces equipment may find the process to be too time consuming and may not comply with therapy recommendations. Improving efficiency in scheduling of rehabilitation therapy progression therefore may be critical to improving patient rehabilitation outcomes.

In addition, another obstacle for providing DAP treatment has been consistency of treatment. Consistency of treatment is especially important in rehabilitation where every session builds upon the improvements of previous sessions. If a patient uses one DAP system at one location for a first treatment, it is then important that the same consistent treatment regime and protocol is applied to subsequent treatments. Currently, the consistency of treatment is largely dependent on the subjective judgment provided by physical therapists. However, even among physical therapy professionals, different treatments may be prescribed for the same patient and the same treatment protocols performed at different times are subject to variability. As such, there is a need for a system and method that allows a user to go to any DAP system and obtain a consistent treatment for the user's specific needs regardless of whether the same facility, DAP system, or physical therapist is involved.

Furthermore, patient compliance has also been a challenge in administering DAP therapy. Because users typically rehabilitate with a single physical therapist and use a single DAP system near their home for treatment, continued treatment becomes a problem when users travel or if work schedules or other considerations require therapy at some distance from the primary physical therapy location. Traveling patients typically do not continue their prescribed treatments because they often do not know if there is an available and appropriate DAP system that can provide consistent treatment for them away from their home location and because patients have no way to transfer the knowledge of their individual physical therapist regarding the appropriate next session in their rehabilitation. Therefore, there is a need for system and method for matching users with DAP systems capable of providing appropriate treatment for the users at a location geographically near the user and for tracking user experience, performance, history, treatment objectives and therapy progression in a manner transferrable from one DAP system to other DAP systems or from one form of rehabilitation equipment, such as isokinetic strength training equipment, to other similar equipment. Such a system would enable a user while traveling or otherwise unable to visit their primary clinic to visit another rehabilitation facility with appropriate equipment and to continue therapy with the same parameters at this alternate location.

### SUMMARY OF THE DISCLOSURE

In some embodiments, a method of differential air pressure (DAP) system treatment management is provided. The method comprises providing a user's information, the information comprising at least two of the following characteristics: age, weight, gender, location, desired result, current medical condition, height, lift access requirements, therapist access requirements, therapy history, past workout information, and user type, wherein user type comprises at least one of an athlete, a casual user, a rehabilitation user, and a chronic user; analyzing, using a processor, the user's information based, at least in part, on aggregate information in a database comprising other users' characteristics and associated past workout session data including duration, speed, incline, and unweighting level used during workouts; and generating, using a processor, a suggested workout routine including duration, speed, incline, and unweighting level to be used during a workout based on the comparing of the user's information to the other users' information.

The analyzing can comprise comprising matching user characteristics to other users' characteristics. Providing the user's information can comprise prioritizing at least one of the characteristics. The matching step can further comprise a.) determining whether at least a portion of the user's characteristics matches at least a subset of at least one user's of the other users characteristics; b.) omitting a lowest priority characteristic from the at least a portion of the user's characteristics to create a prioritized user information set if step a produces no match using the at least a portion of the user's characteristics; c.) determining whether the prioritized user information set matches at least a subset of at least one user's of the other users characteristics; and d.) repeating steps b and c until the prioritized user information matches at least a subset of the at least one user's characteristics. In some embodiments, analyzing comprises identifying at least one other user sharing characteristics with the user and having a favorable workout outcome. The favorable workout outcome can comprise at least one of user satisfaction, obtaining the desired result and progress towards the desired result. Current medical condition can comprise at least one of original diagnosis, dates of injuries, date or type of illness, date or type of interventions, an indication of rehabilitation progress, and a previous treatment and date of treatment. In some embodiments, therapy history comprises prescribed therapy history, actual therapy history, therapy history on a DAP system, therapy history using other equipment. The method can further comprise generating a recommended therapy or workout based on a medical guideline. In some embodiments, providing the user's information occurs at a same appointment or workout session as the analyzing and generating steps. In some embodiments, providing the user's information occurs at an earlier appointment or workout session as the analyzing and generating steps. Providing the user's information can comprise creating a user profile or presenting a unique identifier. The method can further comprise sending the suggested workout routine to a medical professional or insurance provider for approval. The method can further comprise modifying, by the medical professional or insurance provider, the suggested workout routine. In some embodiments, the generating step comprising generating more than one suggested workout routines. The method can further comprise transferring funds from the user to a treatment facility or provider. The method can further comprise providing a cost for the suggested workout routine. Differential pricing can be used to determine the cost. The method can further comprise providing a list of DAP systems appropriate for the suggested workout routine. The method can further comprise providing available appointment times for suitable DAP systems. The method can further comprise scheduling an appointment. In some embodiments, generating a suggested workout routine comprises generating workout routine on equipment other than a DAP system. The method can further comprise uploading the suggested workout routine to the database. The method can further comprise performing the suggested workout and uploading performance data to the database. In some embodiments, the method comprises an iterative process, generating periodic updates for the user or a medical professional. The method can further comprise generating subsequent suggested workout routines based on user progress.

In some embodiments, a system for DAP usage management is provided. The system comprises a storage database comprising past user information and related workout data; a user interface allowing a present user to access information from or add information to the storage database, the information comprising at least two of the following characteristics: age, weight, gender, location, desired result, current medical condition, height, lift access requirements, therapist access requirements, therapy history, past workout information, and user type, wherein user type comprises at least one of an athlete, a casual user, a rehabilitation user, and a chronic user; a processor comprising instructions for comparing present user information and past user information and related workout data and generating a suggested workout routine including suggested duration, speed, incline, and unweighting to be used during a workout based on the comparing of the present user information to the past user information and related workout data.

The system can be configured to connect to one or more DAP systems. The storage database can comprise a centralized or cloud based database. In some embodiments, the user interface can be accessed through a network interface such as an internet or LAN, a local terminal, laptop, tablet, computer, or smart phone. The system can comprise instructions for sending the suggested workout routine to a particular DAP system, a medical professional, or an insurance provider.

In some embodiments, a method of finding an available and appropriate DAP system site is provided. The method comprises identifying a user; providing a user location; providing one or more user system characteristics to identify an appropriate DAP system, the user system characteristics comprising at least one of a user type, the user type comprising at least one of an athlete, a casual user, a rehabilitation user, and a chronic user, a medical condition, a desired result, and a DAP system access need; matching, using a processor, the user system characteristics with one or more appropriate DAP systems based on DAP system features comprising type of DAP system, unweighting provided, access provided, and analysis capability; and generating, using a processor, one or more suggested DAP system sites based on compatibility of the DAP system sites with the user location and the one or more appropriate DAP systems.

In some embodiments, access needs comprises at least one of a need for lift assistance and need for a physical therapist on site. In some embodiments, determining one or more appropriate DAP systems comprises determining one or more appropriate DAP systems having the most DAP system features compatible with the user system characteristics. Compatibility of a DAP system site with a user location can be based on proximity of the DAP system site to the user location. The method can further comprise providing additional user requirements, the additional user requirements comprising desired time slot, desired day of the week, and insurance requirements. In some embodiments, generating one or more suggested DAP system sites is based on availability of the additional user requirements at the one or more appropriate DAP systems and with proximity of the one or more appropriate DAP systems to the user location. The generating step can comprise providing a list of suggested DAP system sites sorted with the site having features matching the highest number of criteria including the additional user requirements, the user location, and the one or more appropriate DAP systems higher than sites having features matching a lower number of criteria including the additional user requirements, the user location, and the one or more appropriate DAP systems. The method can further comprise prioritizing criteria including the additional user requirements, the user location, and the one or more appropriate DAP systems. In some embodiments, a criterion related to the one or more appropriate DAP systems is a highest prioritized criterion. The generating can comprise a) determining whether at least a portion of criteria including the additional user requirements, user location, and the one or more appropriate DAP systems match at least a subset of features of a DAP system site; b) omitting a lowest priority criteria from the at least a portion of criteria including the additional user requirements, the user location, and the one or more appropriate DAP systems to create a prioritized criteria set if there is no match using the at least a portion of the criteria; c) determining whether the prioritized criteria set matches at least a subset of features of a DAP system site; and d) repeating steps b and c until the prioritized criteria set matches at least a subset of features of a DAP system site. Providing user system characteristics can comprise providing at least one of a desired result or medical condition. The matching step can further comprise comparing the at least one of a desired result or medical condition with past workout data of other users' having a same desired result or medical condition and determining one or more suggested workouts based on the comparing. The matching step can further comprise determining DAP system sites capable of providing the one or more suggested workouts. In some embodiments, the suitable DAP system sites change over time as the user progresses towards a goal or in recovery. The method can further comprise scheduling an appointment for the user at a particular DAP system site. The method can further comprise creating a workout protocol or modifying pre-programmed workout protocols and attaching the protocol to the appointment. Attaching the protocol to the appointment can override any system-generated protocol. The method can further comprise providing the user's information, the information comprising at least one of the following characteristics: age, weight, gender, location, desired result, current medical condition, height, lift access requirements, therapist access requirements, therapy history, past workout information, and user type, wherein user type comprises at least one of an athlete, a casual user, a rehabilitation user, and a chronic user; analyzing the user's information based, at least in part, on aggregate information in a database comprising other users' information and associated past workout session data including duration, speed, incline, and unweighting level used during workouts; and generating a suggested workout routine including duration, speed, incline, and unweighting level to be used during a workout based on the comparing of the user's information to the other users' information. The method can further comprise allowing payment for a future appointment.

In some embodiments, a system for finding an available and appropriate DAP system site is provided. The system comprises a user interface for providing a user location and one or more user system criteria to identify an appropriate DAP system, the user system criteria comprising at least one of a user type, the user type comprising at least one of an athlete, a casual user, a rehabilitation user, and a chronic user, a medical condition, a desired result, and a DAP system access need; a processor comprising instructions formatching the user system criteria with one or more appropriate DAP systems based on DAP system features comprising type of DAP system, unweighting provided, access provided, and analysis capability, and generating one or more suggested DAP system sites based on compatibility of the DAP system sites with the user location and the one or more appropriate DAP systems. The system can comprise a database of aggregate user information and related workout data. The system can be connected to one or more DAP systems. In some embodiments, an access need comprises at least one of a need for lift assistance and need for a physical therapist on site. In some embodiments, the user interface is configured for providing additional user requirements, the additional user requirements comprising desired time slot, desired day of the week, and insurance requirements. The processor can comprise instructions to match the one or more appropriate DAP systems with the additional user requirements.

In some embodiments, a method of using a DAP system is provided. The method comprises downloading a workout routine to a DAP system, the workout routine comprising a desired duration, speed, incline, and level of unweighting; identifying a user to the DAP system; performing the workout routine; and recording performance data during the workout routine in the DAP system. The method can further comprise connecting the DAP system to a network. The method can further comprise uploading the performance data to the network. The method can further comprise providing user or therapist feedback to the DAP system. User feedback can comprise feedback regarding at least one of satisfaction with the workout routine, overall mood and level of pain. Therapist feedback can comprise at least one of observations of the workout routine and rating of user progress. In some embodiments, identifying the user comprises providing user information or providing an identifier configured to access user information through the DAP system. An appropriate workout routine can be selected based on user information. In some embodiments, the appropriate workout routine is selected based on reviewing past workout routines and performance data of other users sharing one or more user characteristics. The method can further comprise adjusting the downloaded workout routine. The method can further comprise sending performance data to at least one of a doctor, and insurance provider, and a patient file. The method can further comprise sending at least one of performance data, user feedback, and therapist feedback to an aggregate user database. In some embodiments, the method further comprises adjusting future DAP workouts based on the performance data, user feedback, or technician feedback. The method can further comprise assessing user performance after a workout session to determine whether to modify workout parameters or scheduling.

In some embodiments, a DAP usage system is provided. The system comprises a DAP system; a user interface configured to allow identification of a user to the system; and a processor comprising instructions for downloading a workout routine to the DAP system, the workout routine comprising a desired duration, speed, incline, and level of unweighting, and recording performance data from the workout routine in the DAP system.

In some embodiments, the system is connected to a network. The user interface can be configured to allow input of user or therapist feedback. User feedback can comprise feedback regarding at least one of satisfaction with the workout routine, overall mood and level of pain. Therapist feedback can comprise at least one of observations of the workout routine and rating of user progress. The system can be connected to a database comprising aggregate user information and related workout data.

In some embodiments, a category 1 DAP is provided. The system comprises a positive pressure chamber with a seal interface configured to receive a portion of a user's body and form a seal between the user's body and the chamber, wherein the system is appropriate for use by users requiring no assistance to use the system.

In some embodiments, a category 2 DAP system is provided. The system comprises a positive pressure chamber with a seal interface configured to receive a portion of a user's body and form a seal between the user's body and the chamber, wherein the system is appropriate for use by users requiring moderate assistance to use the system.

In some embodiments, a category 3 DAP system is provided. The system comprises a positive pressure chamber with a seal interface configured to receive a portion of a user's body and form a seal between the user's body and the chamber, wherein the system is appropriate for use by users requiring full assistance to use the system.

In some embodiments, a method of finding an available and appropriate DAP system site is provided. The method comprises identifying a user; providing a user category, the user categories comprising category 1, comprising users requiring no assistance, category 2, comprising users requiring moderate assistance, and category 3, comprising users requiring full assistance; and matching, using a processor, the user to one of a plurality of categories of DAP systems based on appropriateness of the DAP category to the user category.

In some embodiments, a method of finding an available and appropriate DAP system site is provided. The method comprises identifying a user; providing a user location; providing a user category, the user categories comprising category 1, comprising users requiring no assistance, category 2, comprising users requiring moderate assistance, category 3, comprising users requiring full assistance; matching the user to an appropriate DAP system category comprising one of a plurality of categories of DAP systems based on appropriateness of the DAP system category to the user category; and generating, using a processor, one or more suggested DAP system sites based on proximity of a DAP site to the user location and availability of the appropriate DAP system category at a DAP site.

In some embodiments, providing a user category further comprises providing at least one of a user type, the user type comprising at least one of an athlete, a casual user, a rehabilitation user, and a chronic user, a type of medical condition, a desired result, and a DAP system access need, the DAP system access needs comprising a need for lift assistance and a need for a physical therapist; and matching, using a processor, the at least one of the user type, the type of medical condition, the desired result, and the DAP system access need to a user category. The method can further comprise matching, using a processor, the at least one of the user type, the type of medical condition, the desired result, and the DAP system access need to a user category. The matching step can comprise matching the at least one of the user type, the type of medical condition, the desired result, and the DAP system access need to a DAP system category.

In some embodiments, a method of finding an available and appropriate DAP system site is provided. The method comprises identifying a user; providing a user location; providing a DAP system category, the DAP system categories comprising category 1, comprising systems providing no assistance, category 2, comprising systems providing moderate assistance, category 3, comprising systems providing full assistance; and generating, using a processor, one or more suggested DAP system sites based on proximity of a DAP site to the user location and availability of the appropriate DAP system category at a DAP site.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIGS. 1A-C are illustrations of embodiments of DAP systems described.

FIG. 2 is a block diagram of an example of a treatment management and scheduling system according to one embodiment.

FIG. 3 is a flow diagram illustrating another embodiment of treatment management and scheduling system.

FIG. 4 is flow diagram illustrating one embodiment of scheduling a user to use an appropriate and available DAP system.

FIG. 5 is a flow diagram illustrating one embodiment of scheduling a user to use an appropriate and available DAP system with a patient and therapist feedback feature.

### DETAILED DESCRIPTION

There are available differential air pressure systems suited to training users or patients in different categories based on a number of factors such as, for example, patient ability to access the machine, the specific training needs of the patient and the physical capabilities of the patient as well as whether the patient requires assistance during training and if so to what degree. For purposes of discussion, three basic categories will be used. Category 1 is healthy athletic with no assistance required. Category 2 is moderate assistance (post-surgical recovery) where the patient can stand in the system with assistance and remain upright. Category 3 patients require a high level of or full assistance, for example assistance for ingress/egress as well as support during therapy. A number of differential air pressure systems for various levels of patient assistance before, during or after use are described in the non-provisional patent application entitled “Differential Air Pressure Systems and Methods of Using and Calibrating Such Systems for Mobility Impaired Users” application Ser. No. 13/423,124 filed on Mar. 16, 2012 (“the '124 application”). The entirety of this application is incorporated herein by reference.

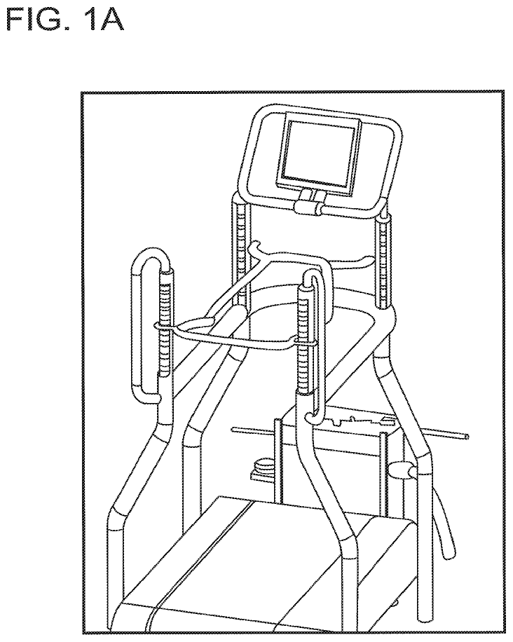

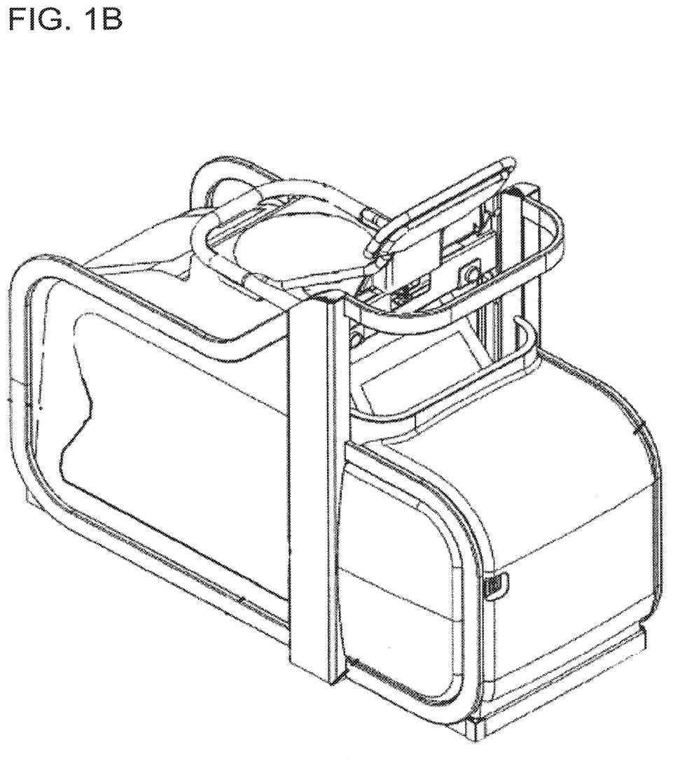

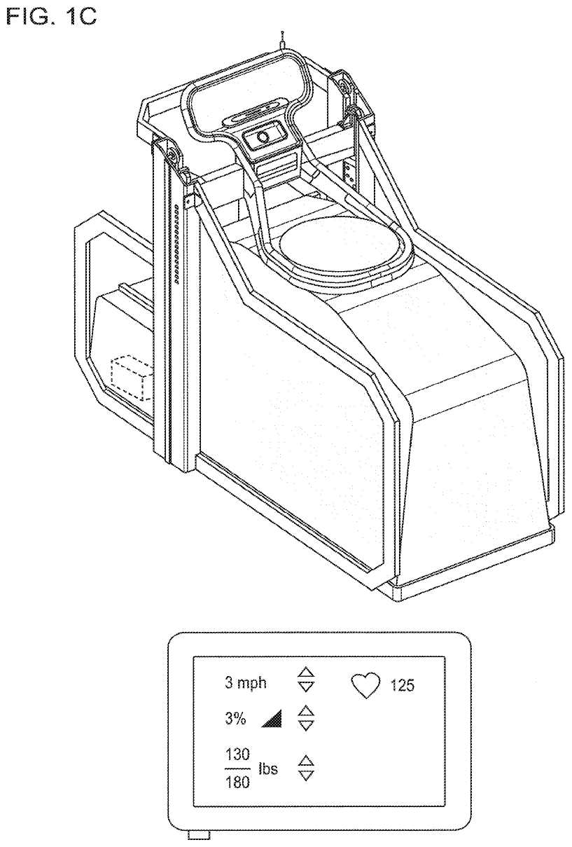

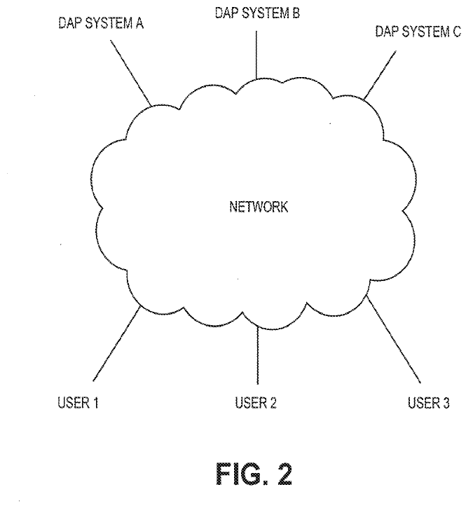

First, with an understanding of the different types of differential pressure systems available, the patient type to use the system, and the desired therapy to be performed, select an appropriate system to perform therapy with a user. A number of systems types for categories 1, 2 and 3 are provided in the '124 application, U.S. application Ser. No. 12/761,316, and U.S. application Ser. No. 12/778,747, the disclosures of which have all been incorporated herein in their entireties. A category 1 system includes for example FIG. 2A of the '124 application, and FIGS. 11 and 16 (shown here as FIG. 1A) of the '316 application. Category 1 systems can be used by healthy, athletic users, and provide a minimal or no amount of assistance for use of the system. A category 2 system includes for example FIG. 7A of the '124 application, and FIGS. 11A (shown here as FIG. 1B) and 11B of the '747 application. Category 2 systems can provide moderate assistance to users, for example, by providing height adjustment mechanisms, lift mechanisms, and counterbalance systems to assist with entry into and stability during use of the DAP system. A category 3 system includes for example FIG. 1A and 19 of the '124 application and 1C of the present application. A category 3 DAP system can provide a high level of assistance and be appropriate for users having low mobility. For example, category 3 systems can include lift systems, including overhead suspension systems with a harness and wheels able to lift a user onto the system. A category 3 system can include a ramp system where a wheelchair can be rolled into proximity of an opening in the DAP chamber.

DAP systems generally utilize a chamber for applying differential air pressure to a portion of a user's body. These systems are especially beneficial in helping users with impaired mobility to regain motor control. For example, a patient unable to bear his full weight upright on his legs under normal weight bearing conditions can use a DAP system to offload a portion or all of his weight. FIG. 1C shows an example of a DAP system having a pressurizable chamber that can be used to offload a user's weight. Once a user enters the DAP system, the chamber is sealed around a portion of the user's body. In some embodiments, the user's body from the waist down is sealed within the chamber. Then, the chamber is positively pressurized to provide an upward force against the user's body in the sealed chamber. The upward force offsets the user's body weight in the chamber. This upward force unweights the user such that the user supports or bears less than his full body weight in the chamber. For example, the pressure may be set to offload the user such that the user supports only 40% of his body weight in the chamber. Once weight is offloaded, a user who may not be able to bear full body weight upright in normal ambient conditions may be able to stand substantially upright in the DAP system and bear a portion of his body weight in the DAP system. A patient whose gait is adversely affected when bearing their full body weight may be able to walk with a more normal gait when unweighted.

As mentioned earlier, DAP systems, especially those with exercise capability, can be beneficial for users who need to or wish to exercise or even stand upright but cannot do so under normal conditions while bearing full body weight. In order to use these systems, a user must first be able to access the chamber, which may require stepping or climbing over one or more portions of the system. In some instances, an individual may have limited or low degree of mobility which may hinder his ability to access the chamber. For example, patients who have suffered a stroke or physical injury may be wheelchair-bound or bedridden and unable to walk or stand independently without a great deal of assistance. Similarly, patients who have a lesser degree of impairment such as muscle strain or a sprain may also require a moderate amount of assistance to enter, stand in, and exit the chamber. Accordingly, these patients with varying levels of impaired mobility may not be able to take advantage of the many benefits of differential air pressure therapy because of the difficulty in getting in and out of the systems. As such a variety of DAP systems have been employed to cater to the needs of specific types of patients. For example, DAP systems described in co-pending International Application No. 61/454,432 filed on Mar. 18, 2011 and U.S. application Ser. No. 13/423,124 filed on Mar. 16, 2012 (hereby incorporated by reference) provides for DAP systems with varied degrees of access and lift assist for accommodating patients with impaired mobility.

However, not all users need access assist and not all DAP systems have access assist. For example the categories of users who may use DAP systems non-exhaustively include: (1) people in need of rehabilitation from an illness or accident; (2) people who want to develop and maintain greater levels of health; (3) people with chronic illnesses, such as Parkinson's Disease; and (4) athletes, who wish to train at high levels of exertion without trauma. As can be appreciated, each user is different, depending on their desired goal, their current state of health, and any prior use of a DAP system. Some of the users have prior experience with DAP systems, while others do not. Some are early in their treatment plan, some are not (and some do not have a treatment plan). Some are elite athletes, some are normal healthy people (for their age group), some are recovering from an injury or illness, and some are chronically ill. Some require additional equipment/assistance to use the machine as required. As such, some embodiments of the current invention provide for a system and method for matching users with the appropriate DAP systems for the needed treatment. In particular, some embodiments provide for a treatment scheduling and management system that accounts for the particular needs of the individual making the appointment for use of a DAP system, beyond simply distance and available time slots. Some embodiments of this invention provide for a system that can schedule multiple therapy activities including sessions on a DAP system as appropriate for the patient, and other therapy sessions for physical manipulation, strength training, electrical stimulation therapy, or proprioceptive or other therapy training to facilitate the rehabilitation and therapy progress, progress of which may be monitored if needed on a device, such as a DAP system, with sensor capabilities. Further, the scheduling system for rehabilitation may be designed to match user requirements with other appropriate rehabilitation equipment in a rehabilitation setting. Some forms of equipment may be appropriate for certain users based upon that user's needs and capacity to use the equipment, such as a bicycle or strength training machines. The system may be designed to understand and distinguish appropriate from inappropriate equipment based upon the user's therapy targets, past workouts, health status, physical limitations and other parameters.

FIG. 2 is a block diagram of an example of a treatment management and scheduling system. As shown in FIG. 2, the treatment scheduling and management system can include one or more DAP systems. In operation, multiple users can be scheduled and paired with appropriate and available DAP systems for treatment over a network. In some embodiments, the systems incorporate (DAP) treadmills capable of bi-directional communication with a central server via a wired or wireless connection. The systems are also capable of operating without a persistent connection, by storing data locally and transmitting when a connection becomes available. In FIG. 2, the DAP Systems A, B, C can communicate bi-directionally with the network to receive and send information regarding a user's treatment during a DAP session. FIG. 1 shows a DAP system with a wireless antenna for wireless connection with a network. Similarly, FIG. 1 also shows the DAP system with a wired connector for wired connection capability. The connection for wireless or wired communication can be placed on any suitable location on the DAP system. The DAP system can also be connected with wires or wirelessly to other rehabilitation equipment for integration of data from those systems and for schedule and treatment parameter management on those systems.

The network can be connected in many different types of architectures including client-server architecture, a peer-to-peer network, or any other type of architecture. The network may include a server that coordinates the comparison of the user's needs with the available DAP systems and the features of those available systems for an appropriate match. The network can also be different types of networks such as the Internet, a local area network, a wide area network, an intranet, an extranet, or a wireless network. In some embodiments, the user may connect to the network via an internet enabled terminal such as an internet connected computer. In other embodiments, the user may connect to the network via a wireless device such as a smart phone or tablet and through a mobile application on the device. The user interface on a computer, phone or tablet may also provide feedback to the user on that user's personal performance metrics and progress, can provide a user access to his own workout data, including video and gait metrics, and can interact with the user to set that user's desired objectives or integrate objectives from the therapist or physician allowing the user to agree with or modify objectives for future treatment sessions thus interacting with the DAP system in scheduling the appropriate therapy and providing inputs and parameters for the next DAP workout when the user is next on the DAP machine or other connected physical therapy device controlled or monitored by the network.

The DAP systems may include an internet or wireless interface that allows the DAP systems to communicate with the network directly or via a computer. For example, the DAP systems may include one or more ports and interfaces to enable a communication line connection through existing broadcast technology, including television broadcast over the airwaves, cable or cable modems, satellite, telephone lines, whether analog or digitally based, the internet, DSL, G-Lite, wireless technology, infra-red (IR) technology, other high-speed data connections, or any other suitable transmission technology or medium. Optionally, a communication port on a DAP system may enable communication directly with another DAP system (such as in a master/slave scenario), whether or not such communication utilizes a network. FIG. 1 provides an exemplary DAP system with bi-directional communication capability. In particular, the DAP system has a communication port that allows the DAP system to communicate with a network or other DAP system.

In some embodiments, the scheduling and management system includes a storage medium, such as a storage database, in the network that is configured to store user information and treatment data as well as information regarding DAP systems connected to the network. The storage medium is designed to store user information such as the user's historical treatment information, contact information, medical information, etc. A storage medium may represent one or more devices for storing data, including read-only memory (ROM), random access memory (RAM), magnetic disk storage mediums, optical storage mediums, flash memory devices, and/or other mediums for storing information.

In some embodiments, the storage medium stores a user created account. Each user of the networked DAP system has an associated secure profile, which contains, for example:
Name
Contact information (Address, City, State, ZIP Code, Country, Phone, Fax, E-Mail)
Billing Information (Credit Card Number, Name on Card, Expiration Date, CVV)
Age
Height
Weight
Medical Conditions (e.g. ICD-9 or CPT4) and dates of injuries, illness, and interventions
Other requirements (e.g. lift access)
Desired treatment objectives—walking improvement, limb strength, balance, or other
User Type (e.g. athlete, casual, rehab, chronic)
Workout or Therapy History (prescribed, actual, on DAP, also on other equipment as appropriate)
Duration, Speed, Incline, Effective Body Weight, Heart Rate, Etc
Appointment Information (past, future)
Payment History

In some embodiments, the DAP systems include a processor and a storage medium. The storage medium of the DAP system records information regarding a user's therapy session on that particular DAP system or on other therapy equipment connected to or controlled by that DAP system. The processor can communicate that stored session information to the network for storage on the network's storage medium. For example, in the situation where user 1 uses DAP system A for a first session treatment, the DAP system A can include a storage medium and a processor where the processor directs the DAP system's storage medium to store the user's session information, including data regarding the user's performance or the physical therapist's notes regarding a user's performance. Once user 1 has completed his session, the processor can communicate the stored information from the storage medium in the DAP system to the storage medium in the network. The network can then retain historical information regarding the user's treatment from DAP system A. Then, in subsequent treatment sessions, the DAP system being used at that time can retrieve the historical treatment information for the user from the network storage medium, import that information into the DAP system's storage medium, and process the treatment information to provide the user with the same treatment protocol.

As an example, if user 1 in a subsequent treatment session is assigned to DAP system B, the scheduling management system can send user 1's previous treatment parameters from DAP system A to the processor of DAP system B. The processor of DAP system B can then implement the treatment parameters from user 1's earlier session on DAP system A. In some embodiments, the same treatment parameters are employed from DAP system A to subsequent DAP system B. In other embodiments, the treatment parameters are changed according to the progress of the user's treatment on the previous device. For example, on DAP system A, a user 1 may be prescribed by a medical professional to have at least ten treatments in a DAP system where the starting treatment has the user support 40% of his body weight in the DAP system. In subsequent treatments, the body weight experienced by the user is gradually increased until the user is able to support 95% of his body weight. The treatment scheduling and management system can store this type of prescribed treatment and provide the appropriate treatment parameters to the DAP system. A user could alternatively be prescribed by a medical professional to use the DAP system and/or other rehab equipment to achieve certain target parameters, such as independent full body-weight walking at 2 miles per hour. The DAP system could use such a therapy target to develop treatment protocols based upon user inputs and feedback such as the level of unweighting required to achieve pain-free walking. The DAP system can then progress the user in subsequent sessions to walking at greater percentage of body weight to achieve gradual improvement toward the desired therapeutic target. Similar patterns of therapeutic target and starting point assessments could be developed for additional therapeutic equipment to measure for example hamstring strength following injury or surgery and then progressively load the hamstring in successive sessions to regain strength toward a therapeutic target.

As an example of integration of multimodal therapy, if a user requires strength training of a lower limb and uses a bicycle or strength training machine in a rehab facility for such training, the system can be electronically linked with that equipment to track the strength improvements and can adjust workout parameters on the DAP system integrating progress on the strength equipment into such DAP workouts. Further if more than one form of strength or training equipment is used, the treatment schedule management system can store the progressive improvement across multiple machines and thereby can both track improvement and can recommend prescribed treatment or record starting or ending points for future treatments across the multiple points of therapy with multiple machines of various types. The integration of these other modes of treatment can be used to complement therapy on a DAP system to enhance overall patient therapy. Gait metrics and other biomechanics evaluation on the DAP system can be used as evaluative markers to assess progress as needed on other rehab modalities.

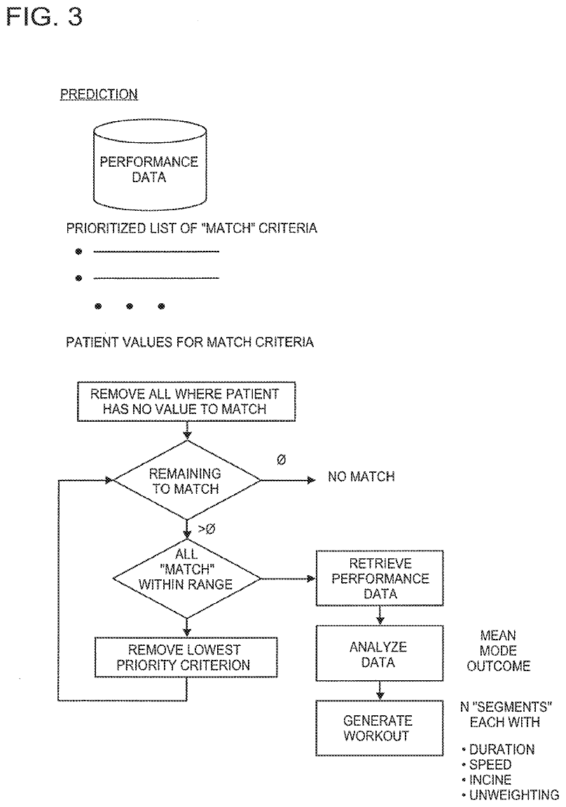

FIG. 3 is a block diagram illustrating an example of one embodiment of a treatment management and scheduling system in which a patient can be scheduled for treatment on an appropriate and available DAP system. In some embodiments, user information is received and stored in a centralized database or a cloud based database. The database may be housed in a server connected to the network. The user can input information for storage into the database through a network interface such as an internet or LAN, or, alternatively into a local terminal, computer, or smart phone or device designed to receive and send the user's information to the networked database.

In some embodiments, the system receives user input such as the user's information. The user's information may include, among other things, the user's name and current zip code. The treatment management and scheduling system receives the user's information and runs a search to determine whether the user has a user profile in the database. If so, the user's information is retrieved from the database. The system then runs a matching algorithm to evaluate the user's criteria and match the user with the appropriate and available DAP systems. The system may implement a matching algorithm to receive a set of treatment criteria A-D from the user to evaluate. The treatment criteria can be obtained either directly in real-time from the user or from historical information stored in the system database. For example, treatment criteria A may be that the user is a paraplegic; treatment criteria B may be that the user is located in Los Angeles; treatment criteria C may be that the user will need a physical therapist on site to evaluate the user's progress; treatment criteria D may be that the desired time slot of 2 pm on a weekend, and treatment criteria E may be related to the patient's insurance provider and participation of certain clinics in or out of certain networks. The system runs an algorithm to compare treatment criteria A-E with the available DAP systems. The algorithm may comprise the steps of going through each of the criteria and assessing which of the DAP systems match the most criteria. For example, a DAP system X may match criteria A, treatment criteria B (being close by in Los Angeles), and treatment criteria D for time slot. However, the DAP system Y may match only on criteria A and criteria D. In that situation, the algorithm would rank DAP system X higher in the option list for the user compared to the DAP system Y.

In additional embodiments, one or more of the treatment criteria may have a higher priority compared to others. In the above example, criteria A for a DAP system capable of use with a paraplegic is higher priority than criteria B for location. This is because the user may need lift assistance to access the DAP system, which may not be available at a DAP system that is closer to the user's location. As such, the algorithm employed by the system can prioritize the treatment criteria such that certain criteria, such as criteria A must match in order for an available DAP system to provide the appropriate treatment for the user. In some embodiments, the algorithm is a predictive algorithm which permits the treatment management and scheduling system to recommend session parameters, based on both the individual's historical performance record as well as other users who share similar characteristics such as original diagnosis, age, gender, and rehabilitation progress. Further, the system may over time modify the recommendation of a DAP system or permitted range of DAP systems or other equipment applicable to a user based upon historical performance parameters. A user who first requires lift assistance and 80% unweighting, may reach a point of exercising at only 40% unweighting and may then be able to use more machines—some of which might be closer to the user's home or may have other advantages on the selection criteria.

In other embodiments, the algorithm is provided in the form of software compatible with a processor in the network. The processor can run the software to evaluate user criteria with available DAP systems.

In addition, to check on the availability of DAP systems, the treatment management and scheduling system may also communicate with network capable DAP systems to determine availability of the DAP systems. In some embodiments, the system provides real time information on the state of every machine, whether the machine is on, in use (and by whom), performance and patient evaluation data (e.g. speed, incline, unweighting, heart rate, gait), and system diagnostic information (e.g. total time, belt alignment, motor current). In such cases, the DAP systems may include a storage medium (as described above) which stores DAP system information such as status of the DAP system and scheduled uses for the system. For example, the DAP system's storage medium may include the scheduled treatments and maintenance for the DAP system. When the treatment management and scheduling system communicates with the DAP system, the treatment management and scheduling system evaluates the status of the DAP system. For example, the treatment management and scheduling system may check if the DAP system is in use or will be in use in the desired time slot. Similarly, the treatment management and scheduling system may evaluate whether the DAP system will be fully operational at the desired time slot.

Once the treatment management and scheduling system has received user information such as the user's name and zip code, the system may then compare the zip code information with stored information on the location of DAP systems. The treatment management and scheduling system may then evaluate which of the DAP systems are suitable for further evaluation based on location and distance from the user. Once a set of potential DAP system candidates are determined based on location, the treatment management and scheduling system may then evaluate the suitability of the DAP system candidates on other user treatment criteria such as whether the DAP system is available for a desired time slot, whether the DAP system has a lift assist device, whether the DAP system has gait training analysis, etc. Based on a comparison of the user's treatment criteria and the available DAP system candidates, the treatment management and scheduling system provides a list of DAP systems from which the user can select. In some embodiments, the DAP systems provided to the user are in descending order of suitability where the first option may match on more criteria or on more important criteria than the last option.

In an alternative embodiment, as shown in FIG. 3, the treatment management and scheduling system may first receive a user's zip code from a website that sends the user's information to the treatment management and scheduling system. The system then provides the user a list of facilities based on distance from the user's location. The user then selects a facility based on the distance. Once the facility is selected, a calendar is provided for the user to select a date and time desired for treatment. The user then enters information such as the user's name and payment information. Once submitted, the treatment management and scheduling system will run the transaction and notify the user of the appointment. The system will also notify the facility of the appointment. The system may then send an additional email to the user requesting additional user specific information such as reasons for appointment, health issues, or other information that is forwarded to the provider/facility. In some embodiments, the confirmation email to the user has the request for additional information.

In some embodiments, the requested user information is provided to the system for processing and comparison with a database of aggregated data from a population of DAP system users. Based on a comparison of the user's information with stored population data, the system may recommend to the user and/or the facility an appropriate treatment regime. For example, the system may receive information from a user indicating that the user is a recent complete knee joint replacement patient. As such, the system may check through its database with a population of users who also have had complete knee joint replacement procedures or may check reference protocols established for typical knee replacement patients based on medical guidelines. The system may then generate a possible treatment protocol based on the types of treatments employed by other users with a similar medical profile or based on medical guideline references. The system's aggregate information may indicate that the majority of complete knee joint replacement patients undergo 20 sessions where each session gradually increases the body weight experienced by the user by 5% and the users generally conduct exercises on a treadmill. Based on that information, the system may suggest that the same or similar treatment protocol would be appropriate for the instant user. That suggestion may be directly provided to the user, medical professional, insurance company, etc. to provide information on suitable treatments. The system may alternatively provide the recommendation based upon medical guideline references, or may provide both benchmarks: the medical guideline range or template protocol and population averages or ranges for similar patients. The system may further provide pre-authorization or other communication with the health insurance provider for the patient and may provide that insurance or other relevant information to the patient and the health care practitioner at the time of patient visit to the clinic to facilitate and streamline the administrative part of the rehab process. The system may further integrate third party information such as insurance authorization for a set number of rehabilitation sessions into the proposed treatment protocol parameters for consideration by the health care practitioner and/or the patient. This authorization cycle and evaluation against population norms or medical guidelines may be an iterative process through the course of treatment with periodic updates provided by the system to the healthcare practitioner and/or the patient. If additional sessions are appropriate for the patient or desired by the patient beyond the insurance authorization, the system in various embodiments may either submit requests and justification for additional insurance coverage or may offer to sell time on the DAP systems directly to the user.

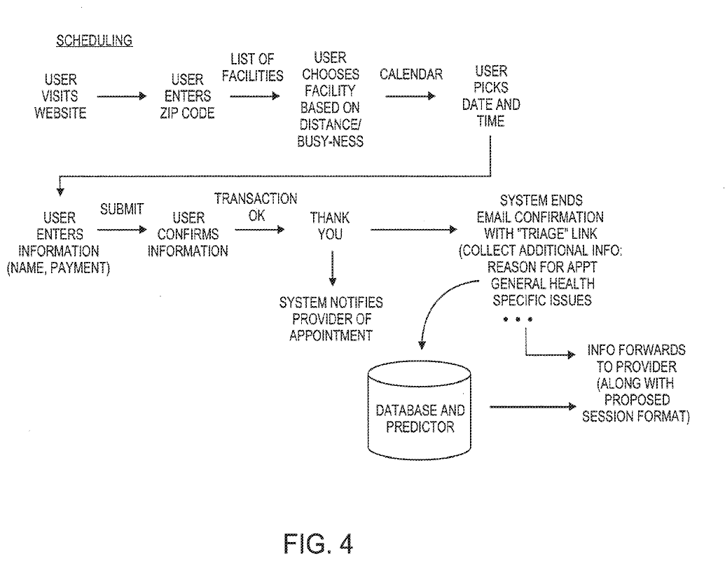

In other embodiments, the user may be matched with a DAP system based on the user's criteria and then the DAP system retrieves the user's previous performance data from a storage medium such as a patient access card or from a networked database. Subsequently, the DAP system analyzes the retrieved data and generates a treatment program appropriate for the user. FIG. 4 provides an example of this embodiment. In this embodiment, the patient provides a list of match criteria to the treatment management and scheduling system. The treatment management and scheduling system evaluates first whether the patient has any match criteria for consideration. Where the patient has no criteria for matching, the system will not run a matching process. Where match criteria are available, the system will compare the criteria with DAP systems available. If all the criteria match, the DAP system(s) will be offered to the patient for use. If some but not all of the criteria match, the treatment management and scheduling system may remove the lowest priority criterion and rerun the matching process until all the criteria match with a DAP system. Once a DAP system is selected, use of the DAP system may be carried out by having the selected DAP system retrieve performance data specific for the user from the treatment management and scheduling system database. The DAP system can then analyze the data and generate a treatment regime for the user. Alternatively, the treatment management and scheduling system may retrieve the user's information and generate a treatment regime that can be received and implemented by a selected DAP system.

In the embodiments where the treatment management and scheduling system generates a suggested treatment regime, the selected DAP system can download the suggested treatment program from the treatment management and scheduling system to run on the selected DAP system. In other words, in some embodiments, the system allows a program for a specific user to be sent to a specific machine for use at a scheduled time. In some embodiments, the DAP system is capable of delivering a specific workout (composed of a set of segments, each with its own duration, speed, incline, and percentage body weight) to the user, based on information sent to it by the treatment management and scheduling system.

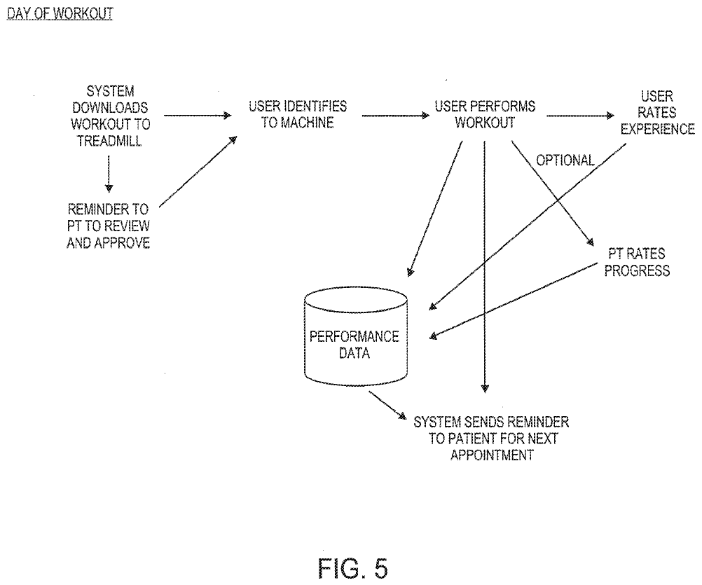

As shown in FIG. 5, the DAP system downloads a treatment or workout program to a DAP system with a treadmill. Either the DAP system or the treatment management and scheduling system may send an approval request to a medical professional or to an insurance provider for approval. For example, a networked DAP treadmill could be pre-set for a workout session based on knowledge of who will be using the machine during that session. A physical therapist could adjust the program locally as required, either prior to or during the session. The system will allow for review and modification of a recommended user program by the associated physical therapist or trainer. For example, in some embodiments, the system allows a therapist to create or modify pre-programmed workout sessions and attach these to an appointment scheduled by the user, overriding any system-generated workout session. In some embodiments, the DAP systems have editing capabilities on a display/control unit associated with the treadmill, or on a mobile device by means of an “app.” In some cases the display or control unit is removable.

Once the treatment is set, the user gets into the DAP system and performs a treatment or workout according to the suggested treatment protocol provided either by the DAP system, the treatment management and scheduling system, the physical therapist, or a combination of these.

In some embodiments, prior to starting the treatment, the user is identified by the DAP system as the proper user for the specific treatment. For example, the DAP system may be capable of identifying the individual user, based on some unique ID which is presented to the machine prior to use. The system will know the age, sex, and medical diagnoses (if applicable) of each user. In some embodiments, the system may require that a user who has scheduled time on a machine to identify themselves to the machine (via keypad, RFID, bar/QR code, magnetic card swipe, biometrics, or other identification technology) at the beginning of their scheduled session. This provides confirmation that the user kept the scheduled appointment, ensures that any treatment protocol sent to the machine is used by the intended user, and allows performance data to be attached to that user's treatment history. Where a patient does not have an identification means, the user can create a profile. The DAP system may maintain a profile of each user. In general, users will identify themselves prior to using the system. In some embodiments, a “guest” identification acts as a catch-all for users without a profile. The system will track utilization by individual users and can report on utilization statistics and workout parameters to the healthcare practitioner for medical evaluation, to the user for personal medical and health records and monitoring, and to third parties such as insurance providers or reimbursement agencies for medical reimbursement to the clinic or healthcare practitioner or for compliance verification of activities by the patient associated with medical insurance or wellness program monitoring.

Advantageously, in some embodiments, a patient identification means can help monitor (and encourage) a patient's compliance with a treatment program. The patient's identification means such as an access card may be read by a medical professional during scheduled checkups to monitor the patient's progress.

Once the user has completed his session, the user can provide feedback to the DAP system. For example, the DAP system can receive and store information on the user's satisfaction with the treatment, overall mood, level of pain, etc. In some embodiments, the DAP system is capable of recording a broad range of information about user performance, including but not limited to duration, speed, incline, percentage body weight, heart rate, and gait factors. Moreover, the DAP system can receive and store information provided by a medical professional observing the user's treatment on the DAP system. For example, a physical therapist may rate the user's progress and/or provide notes on the user's treatment. Any of this information can be directly entered into the DAP system either by a computer terminal interface connected to the DAP system or through a receiving means directly connected to the DAP system. For example a touch pad monitor may be connected to the DAP system to receive input.

The DAP system may also be configured to send information to another device such as a printer or computer. The information can be sent via email to a doctor, insurance company, or a patient file. In other embodiments, the information can be printed and added to a physical file at the facility. Additionally, the information may be sent to the treatment management and scheduling system to be stored in the database for archival and retrieval purposes. For example, the DAP system may be capable of transmitting that information to a central information processing system.

In some embodiments, information is sent to a doctor or insurance company if the treatment protocol indicates that more sessions are required and the user does not have a prescription or insurance coverage for the remaining suggested sessions. In some embodiments, a predictive algorithm is used to evaluate whether a suggested treatment protocol generated by the DAP system or the treatment management and scheduling system is consistent with the prescribed treatment by a medical professional. If, for example, the predictive algorithm shows that the number of covered sessions remaining is less than the number of treatments predicted to achieve the desired outcome, the system (DAP or treatment management and scheduling) will generate a reminder to the facility/therapist that re-authorization is required. The system may also generate the required documentation needed for re-authorization.

In some embodiments, to determine proper scheduling of the appropriate DAP system, the treatment management and scheduling system evaluates criteria besides the machine being used, such as specific therapist or skill set, whether the patient needs assistance in entering or using the machine (including need for lift access), video recording systems, gait analysis capabilities, insurance qualification and provider network, and transportation to/from the appointment.

In some embodiments, the system will use data from gait analysis, user performance, user experience, etc. to drive scheduling. For example, the treatment management and scheduling system may receive and gather a user's information after the first treatment. Based on that information, the treatment management and scheduling system can provide the user with additional sessions or a series of sessions for continued treatment based on the first treatment and the end goal. In other embodiments, the treatment management and scheduling system continuously assesses the user's performance and information after each session to determine whether to modify treatment parameters or scheduling. For example, a user reports that they experienced pain during the appointment, the system may suggest delaying the next appointment, to allow for more recovery time or may recommend a greater degree of unweighting at the next DAP session. If the machine senses gait asymmetry that may be associated with muscle strength, the system may recommend possible strength or flexibility rehab therapies as part of the PT evaluation and possible treatment considerations and the system could monitor compliance with specific recommended activities if such activities are performed on machines connected to the system or if the patient is wearing sensors that enable data capture of such activity when not on connected machines.

In further embodiments, the treatment management and scheduling systems allow a sequence of appointments to be scheduled, based on either a number (e.g. 10 appointments) or a desired outcome (e.g. walking at 3% incline at 2 mph at 95% of body weight). Rather than schedule a single appointment as described, multiple appointments can be scheduled by the user according to desired number of appointments or treatment protocol. The system can monitor patient compliance with the treatment schedule and can monitor patient progress toward the desired outcomes. If necessary, the system can communicate recommended or possible modification to the treatment sessions required. Such communications could be provided to the healthcare practitioner, to the patient, to the insurance provider or to other parties with associated data and rationale based on patient-specific or population data metrics.

In some embodiments, the treatment management and scheduling systems will create a recommended program for a user's next appointment, based on, among other things, the patient's purpose in using the machine, their current medical condition, their historical performance, and aggregate data collected by the system about the performance and progress of other users with similar characteristics. The system may do so by comparing the user's performance data from the last treatment session with aggregated data collected by the system for a population of users. The system may then generate a recommended treatment program for the user's next appointment based on the comparison of the user's information and stats with the data for the population of users.

In some embodiments, the aggregated data may include a performance database based on the demographic and medical data about users and their related workout sessions. This performance database will include and accumulate a qualitative measure from the user about their experience (e.g. pain, satisfaction) during the session. In further embodiments, the aggregated data may include and accumulate data from medical personnel (e.g. physical therapists supervising users) as to the outcome of a user's DAP treatment session. This data will also be stored in the performance database.

In some embodiments, the user may not have any prior experience with DAP systems. In such cases, the systems described can design a suitable DAP treatment based on the user's information. For example, a user with no prior DAP system experience may wish to use DAP to improve the user's running speed. To design the appropriate DAP system, the treatment management and scheduling system may receive the user's information regarding the desired treatment result. In this example, the user may input into the treatment and scheduling system that she wants to decrease the time needed for her to run a mile. The user may optionally input additional information regarding her location and the time slot for the treatment. The treatment and scheduling system then employs a predictive algorithm, such as the ones described above, to determine the appropriate treatment and DAP system for the user. The predictive algorithm may compare the user's information to that in a database with aggregate data (including performance data) regarding the population of users that have used a DAP system. The algorithm then assesses the treatment parameters employed by other users to determine what treatment would be suitable for the user. The treatment management and scheduling system may then provide one or more suggested treatments to the user and have the user decide on a treatment.

In the case where multiple treatment options are available, the user may first decide on the type of treatment. Once that is selected, the treatment management and scheduling system may then determine which DAP systems or other rehabilitation equipment can provide that treatment. For example, if the algorithm determines that users can improve running speed by modifying gait or by running under positive pressure, the system may offer those two treatment options to the user. If strength or flexibility improvement is needed along with use of the DAP system, the scheduling system can recommend treatments involving multiple modes of therapy. If the user picks gait modification as a treatment, the treatment management and scheduling system may then match the user with DAP systems having gait analysis capability. Alternatively, the treatment management and scheduling system may offer the DAP systems to the user and indicate in the listing that the DAP system selected can provide gait or positive pressure treatment.

In an alternative embodiment, the treatment management and scheduling system or DAP system may not utilize aggregate data or performance data from a population of users to determine the suitable treatment protocol for a specific user. Rather, in some embodiments, the treatment management and scheduling system or the DAP system may receive the user's prescribed treatment from the user or medical professional. For example, a doctor may prescribe that a joint replacement patient undergo at least ten sessions with a DAP system. This information is received by either the DAP system or the treatment management and scheduling system. If received by the DAP system, the DAP system will relay the information to the treatment management and scheduling system for the user's profile. The treatment management and scheduling system can then schedule a series of appointments with suitable treatment parameters for the user. For example, the treatment management and scheduling system may schedule a weekly session for ten weeks based on the prescription.

In other embodiments, the DAP system or the treatment management and scheduling system may have pre-programmed sessions designed for commonly requested treatments. For example, if training for a marathon is a common desired treatment, the DAP system or the treatment management and scheduling system may have a pre-programmed training protocol with differing number of sessions depending on user's fitness level. The treatment management and scheduling system can then schedule a series of sessions for the user based on the training protocol.

In further embodiments, the treatment management and scheduling system may use rules for designing or determining an appropriate treatment for a user. These rules may be developed by interviewing subject matter experts (e.g. a “expert system”) such as physical therapists or trainers to create a treatment program for download onto a DAP system. Rules may also be based on the patient diagnosis and variations of a standard protocol based on known data (e.g. age, weight, gender).

Advantageously, with any of the treatment systems and methods discussed, once a treatment and user are scheduled for a DAP system, the DAP system can run the treatment session without human supervision. For example, once a DAP system has downloaded an appropriate treatment session from the treatment management and scheduling system, the DAP system can run the session for the user without a physical therapist's intervention or supervision. Because the appropriate DAP treatment parameters have been previously determined for the user, the DAP system can run the treatment for the user without supervision during the treatment.

In further embodiments, the system will allow the information about a given user, including their recommended “next appointment” workout program to be accessed using any machine/facility which meets the requirements of the program. The intent being to allow users who travel to continue using a DAP system wherever they happen to be.

In some embodiments, the treatment management and scheduling system will provide an interface which allows an individual user access to all the information held by the system about them: personal information, medical information, appointment history, billing and payment history, recommended workouts and actual performance data.

The system (with the user's permission, and using authentication provided by the user [e.g. username, password]) can access other online health data, such as that provided by Microsoft's Health Vault, FitBit, or Nike, and incorporate that information into its knowledge about the user in order to schedule the most effective possible series of appointments for the user.

In some embodiments, the treatment management and scheduling system is integrated with non-DAP systems to capture user treatment data and protocols on non-DAP systems. A user using a DAP system may also use multiple other pieces of equipment in a rehab setting, such as bikes, isometrics measuring devices, strength training, electrical stimulation devices etc. All of the data from these devices can be integrated with progress of rehab in the anti-gravity environment to collectively create rehab benchmarks, protocols etc.

To the extent that other, non-DAP equipment must be scheduled for use by DAP system patients as part of their training or rehabilitation program, that equipment can also be scheduled as part of a single appointment or a series of appointments. That equipment can be connected to the treatment management and scheduling system via a wired or wireless connection, just as with the DAP treadmill. In some embodiments, such equipment could also have a keypad, RFID capability, or other mechanism to allow users to identify themselves before use. As described with the DAP systems above, a machine could return data which would be useful in scheduling the next appointment and workout format. For example a strength machine might return the number of repetitions, the amount of weight, and the user's heart rates. If the machine can operate under computer control (e.g. setting the weight to be lifted on a strength machine), then the treatment management and scheduling system can download a pre-programmed session to the machine. As with the DAP system, the program could be reviewed by a professional before being sent to the machine for the user.

Alternatively, a display on the machine could direct the user on the treatment/workout. In cases (such as fitness training) where professional supervision is not a requirement, the treatment management and scheduling system could schedule, charge for, verify use, control parameters (either directly or via display to the user), record outcomes, and analyze effectiveness of a patient's usage of a DAP treadmill and other non-DAP fitness devices (such as various types of weight machines, elliptical trainers, etc.). The system could also capture this information and provide evidence of treatment compliance along with reports of workout parameters to support insurance reimbursement applications, or the system could automatically submit for reimbursement based upon parameters provided for each user or for each facility.

In other embodiments, the treatment management and scheduling system may account for payments to the owner of the treatment management and scheduling system and/or the facility/provider. For example, the system may split revenue based on the agreement in place with each facility/provider. The system will automatically transfer funds or invoice the facility provider as required.

In some embodiments, the owner of the treatment management and scheduling system and the DAP system facility may agree to split the revenue for all users referred to the facility by the treatment management and scheduling system, for the patient's first 5 appointments. In such cases, the treatment management and scheduling system can be configured to keep track of the patient's use and account for the revenue sharing arrangement. In some embodiments, the revenue sharing arrangement may not be the same for all 5 appointments but rather 50% for the first appointment, 40% for the second, 30% for the third, 20% of the fourth and 10% for the fifth. Additionally, any revenue sharing arrangement may be employed and implemented by the system. In some embodiments, the system attempts to ensure that a user does not generate multiple IDs by comparing information with existing users when a new ID is about to be created. Facilities need to trust that the system is not creating a new ID for an existing customer, thus cutting into their revenue.

In some cases, the revenue sharing arrangement can be characterized by a formula, f(n, r), which shows the amount of money due to the system owner (positive result) or the facility (negative result) as a function of n (representing the n-th appointment) and r (the revenue received for the nth appointment). The formula can be associated with a specific DAP treadmill, rather than the facility, allowing per-machine pricing.

In some embodiments, the treatment management and scheduling system provides differential pricing, based on (among other things) regional pricing differences (i.e. New York City vs. Bakersfield), popularity (i.e. congestion pricing), equipment (e.g. a treadmill which cannot incline vs. one which can) and the likelihood that an appointment slot will go unused (generating no value whatsoever). A differential pricing feature can improve (and maximize) a return on investment for a DAP system that is fully utilized during the days/hours it is available for use. This can be important because once an available time slot has passed without scheduling the DAP system for use, that time slot is valueless.

In some situations, there may be more than one machine at different locations that can meet a user's need to schedule an appointment. These DAP systems may have different features that allow users to conduct varied treatment plans. As such, the treatment management and scheduling system allows these DAP systems to be priced differently. For example, in some embodiments, there is a minimum price set for a given amount of time on a treadmill machine in a DAP system (e.g. price for a 30- or 60-minute session). This minimum price may be different for different DAP systems. There may be different prices for supervised vs. unsupervised use of the DAP systems. DAP systems with gait analysis and training may be priced higher than DAP systems without that feature.

When scheduling a particular machine, the treatment management and scheduling system can price a treatment by assessing several factors. One possible factor is past and/or future frequency of possible appointment times for that machine on the day chosen by the user. For example, if the user is looking for an hour-long appointment at noon on a day when the facility is open from 8 AM to 5 PM, and has the highest demand for appointments at noon, the treatment management and scheduling system can price the noon time slot at a higher cost than other time slots. Additionally, if the facility usually sets half-hour appointments rather than 1-hour appointments, the treatment management and scheduling system can charge the 1 hour appointment twice the cost of usual half-hour session.

In order to price DAP systems and treatment sessions, the treatment management and scheduling system can take all the possible appointment times (day of week, time of day) for a given window and track the appointment times. For example, for a time slot of noon on Tuesday, the system can evaluate how the noon slot has been used in the past for weekdays (Tuesday) in the past and determine pricing of the slot for the future. Past appointments and future appointments may be weighted differently by the system to favor past experience over future trends or vice versa. The system will also consider if some hours during the day are regularly unavailable (e.g. office closed for lunch or excepted from scheduling by the facility where the machine is located). The system may also consider desired parameters set by the facility, for example that no two successive time slots are filled by the system to permit sufficient time in between automatically scheduled slots for last minute in-clinic patients to use the equipment.

In some embodiments, for each appointment time, the treatment management and scheduling system will calculate how many times the spot was actually reserved. The system can also order appointment times from most popular (highest number of reservations) to least popular. The system may then break this list into three sections, such that the total number of reservations is the same in each of the three sections. In other words, the number of appointment times in each section may differ significantly. Each section of the list represents the same amount of revenue if all appointments are priced the same. The section containing the most popular appointment time can be considered as the “popular” section, and the section part containing the least popular appointment time as the “unpopular” section.

In some variations, the system assigns automatically higher prices to appointment times in the “popular” section (premium pricing) and lower prices to appointment times in the “unpopular section”). There might be three prices (one for each section), or finer gradations (up to a distinct price for each appointment time). In some cases, the goal of the pricing algorithm is to adjust pricing so that each of the three sections is the same length or substantially the same length (i.e. represents the same number of appointment times). This can maximize utilization of the machine (assuming all appointments are kept).

Alternatively, a pricing algorithm of the treatment management and scheduling system can adjust pricing so that the total revenue is maximized by making the most popular segment (premium pricing) represent the largest number of reservations possible, and the unpopular segment (discounted appointments) as small as possible.

The treatment management and scheduling system can also maintain the price charged for every appointment made. Since health insurance plans pay a fixed amount, the system may in some embodiments filter out these fixed price appointments prior to calculation.

In operation, when the treatment management and scheduling system presents potential appointment times for a given machine, the system also shows the price for those appointments (which may vary). Users who are motivated by price are likely to sign up for “unpopular” (and thus cheaper) sessions. Users motivated by choice of time are more likely to be willing to pay for a “popular” (and more expensive) time slot. Additionally, users may be willing to change an existing appointment to save money.

Over time, the system can develop optimum pricing models based on actual data. These can be used as the initial defaults for new DAP systems where no pattern has been established.

Additionally, in some variations, for appointments which are not scheduled by some point in advance of the appointment time, the system will review scheduled users of the same machine and, based on factors such as their current appointment time, optimum appointment spacing, and availability of required resources (such as transportation), offer the unscheduled slot to those users on a first-come, first-served basis. Users whose currently-scheduled appointments are more distant in time will be contacted first, since that allows additional time to fill the newly opened future appointment time.

Additionally, the treatment management and scheduling system can account for regional pricing differences by establishing a base price for each DAP system, based on its location.

In a further exemplary implementation, there may also be available to a user a progression of personal assistance, unweighted training and rehabilitative systems along with other non-assistive or conventional exercise systems. This variety of training systems may be considered a continuum of care. An individual may be training to recover from a stroke or surgery. Such an individual may not be able to move without assistance. As such, one of the assistive devices described herein would be used as the starting point for this person's training or rehabilitation program. In one aspect, the user may be provided with an assistive device that in this context refers to a device that may include an actuator or other form of imparting locomotion to the user's limb or frame to assist the user in the biomechanics of walking. In one aspect, there may be one or more actuators coupled to the person's limbs or about one or more joints to aid in moving the person's limbs to provide assisted mobility training. Next, after some sessions and improvements, the person may progress to one of the various unweighting systems or other assistive training systems described herein. After a progression through the stages of assistive training, the person may progress to the use of unassisted training or exercise equipment. In general this continuum of care from fully assisted (alone or in combination with unweighting training) progresses to unweighting types of training. The user may then progress to lesser amounts of unweighting (i.e., the unweighting system provides less and less assistance) as the user gets stronger and more able to accomplish gait and mobility independently. Until the user reaches the use unassisted exercise and independence of gait and other biomechanical training and rehabilitation.

The systems described herein may also be configured to accommodate a user's progress through the above mentioned stages or continuum of care from assistive locomotion devices or systems, to unweighting systems to lesser degrees of unweighting systems to the use of conventional exercise equipment and training systems. In the exemplary descriptions of the implementation of these integrated training systems, the term “training device” is intended to include any of the herein described training systems including assisted locomotion devices or systems or actuator based limb mounted components; non-DAP unweighting systems; DAP unweighting systems or conventional training systems such as treadmills, stationary bikes, elliptical trainers, stair climbers and the like.

Referring again to FIG. 5, the system downloads a treatment or workout program to the appropriate assisted, unweighting or other training device. Either the training system or the treatment management and scheduling system may send an approval request to a medical professional or to an insurance provider for approval. For example, a networked training device could be pre-set for a workout session based on knowledge of who will be using the machine during that session. A physical therapist could adjust the program locally as required, either prior to or during the session. The system will allow for review and modification of a recommended user program by the associated physical therapist or trainer. For example, in some embodiments, the system allows a therapist to create or modify pre-programmed workout sessions and attach these to an appointment scheduled by the user, overriding any system-generated workout session. In some embodiments, the training device or systems have editing capabilities on a display/control unit associated with the treadmill, or on a mobile device by means of an “app.” In some cases the display or control unit is removable.

Once the treatment is set, the user gets into the training device or system and performs a treatment or workout according to the suggested treatment protocol provided either by the training device or system, the treatment management and scheduling system, the physical therapist, or a combination of these.

In some embodiments, prior to starting the treatment, the user is identified by the DAP system as the proper user for the specific treatment. For example, the training device or system may be capable of identifying the individual user, based on some unique ID which is presented to the machine prior to use. The system will know the age, sex, and medical diagnoses (if applicable) of each user. In some embodiments, the system may require that a user who has scheduled time on a machine to identify themselves to the machine (via keypad, RFID, bar/QR code, magnetic card swipe, biometrics, or other identification technology) at the beginning of their scheduled session. This provides confirmation that the user kept the scheduled appointment, ensures that any treatment protocol sent to the machine is used by the intended user, and allows performance data to be attached to that user's treatment history. Where a patient does not have an identification means, the user can create a profile. The training device or system may maintain a profile of each user. In general, users will identify themselves prior to using the system. In some embodiments, a “guest” identification acts as a catch-all for users without a profile. The system will track utilization by individual users and can report on utilization statistics and workout parameters to the healthcare practitioner for medical evaluation, to the user for personal medical and health records and monitoring, and to third parties such as insurance providers or reimbursement agencies for medical reimbursement to the clinic or healthcare practitioner or for compliance verification of activities by the patient associated with medical insurance or wellness program monitoring.

Advantageously, in some embodiments, a patient identification means can help monitor (and encourage) a patient's compliance with a treatment program. The patient's identification means such as an access card may be read by a medical professional during scheduled checkups to monitor the patient's progress. Monitoring progress may also be used to track, monitor, adjust or improve upon a user's progression along the continuum of care as described above.

Once the user has completed his session, the user can provide feedback to the training device or system in any number of ways. For example, the training device or system can receive and store information on the user's satisfaction with the treatment, overall mood, level of pain, etc. In some embodiments, the training device or system is capable of recording a broad range of information about user performance, including but not limited to duration, speed, incline, percentage body weight, heart rate, and gait factors. Moreover, the training device or system can receive and store information provided by a medical professional observing the user's treatment on the training device or system. For example, a physical therapist may rate the user's progress and/or provide notes on the user's treatment, or progression from one assistive device or technique to the next along the continuum of care described above. Any of this information can be directly entered into the device or training system either by a computer terminal interface connected to the device or system or through a receiving means directly connected to the device or training system. For example a touch pad monitor may be connected to the device or system to receive input.

The device or training system may also be configured to send information to another device such as a printer or computer. The information can be sent via email to a doctor, insurance company, or a patient file. In other embodiments, the information can be printed and added to a physical file at the facility. Additionally, the information may be sent to the treatment management and scheduling system to be stored in the database for archival and retrieval purposes. For example, the training device or system may be capable of transmitting that information to a central information processing system.

In some embodiments, information is sent to a doctor or insurance company if the treatment protocol indicates that more sessions are required and the user does not have a prescription or insurance coverage for the remaining suggested sessions. In some embodiments, a predictive algorithm is used to evaluate whether a suggested treatment protocol generated by the training device or system or the treatment management and scheduling system is consistent with the prescribed treatment by a medical professional. In one aspect, the system will also predict or recommend the progression of a user from one type of assisted training device or system to another based on user performance, goals, historical data or one or more factors provided by a predictive training algorithm. If, for example, the predictive algorithm shows that the number of covered sessions remaining is less than the number of treatments predicted to achieve the desired outcome, the system (DAP, non-DAP, training device or system or treatment management and scheduling) will generate a reminder to the facility/therapist that re-authorization is required. The system may also generate the required documentation needed for re-authorization.

In some embodiments, to determine proper scheduling of the appropriate training device or system, the treatment management and scheduling system evaluates criteria besides the machine being used, such as specific therapist or skill set, whether the patient needs assistance in entering or using the machine (including need for lift access or a particular personal training device or locomotion system or gait monitoring system), video recording systems, gait analysis capabilities, insurance qualification and provider network, and transportation to/from the appointment.

In some embodiments, the system will use data from gait analysis, user performance, user experience, etc. to drive scheduling. For example, the treatment management and scheduling system may receive and gather a user's information after the first treatment. Based on that information, the treatment management and scheduling system can provide the user with additional sessions or a series of sessions for continued treatment based on the first treatment and the end goal. In other embodiments, the treatment management and scheduling system continuously assesses the user's performance and information after each session to determine whether to modify treatment parameters or scheduling. For example, a user reports that they experienced pain during the appointment, the system may suggest delaying the next appointment, to allow for more recovery time or may recommend a greater degree of unweighting, or different unweighting system or technique at the next session. If the machine senses gait asymmetry that may be associated with muscle strength, the system may recommend possible strength or flexibility rehab therapies as part of the PT evaluation and possible treatment considerations and the system could monitor compliance with specific recommended activities if such activities are performed on machines connected to the system or if the patient is wearing sensors that enable data capture of such activity when not on connected machines.

In further embodiments, the treatment management and scheduling systems allow a sequence of appointments to be scheduled, based on either a number (e.g. 10 appointments) or a desired outcome (e.g. walking at 3% incline at 2 mph at 95% of body weight). Rather than schedule a single appointment as described, multiple appointments can be scheduled by the user according to desired number of appointments or treatment protocol. The system can monitor patient compliance with the treatment schedule and can monitor patient progress toward the desired outcomes. If necessary, the system can communicate recommended or possible modification to the treatment sessions required. Such communications could be provided to the healthcare practitioner, to the patient, to the insurance provider or to other parties with associated data and rationale based on patient-specific or population data metrics.

In some embodiments, the treatment management and scheduling systems will create a recommended program for a user's next appointment, based on, among other things, the patient's purpose in using the machine, their current medical condition, their historical performance, and aggregate data collected by the system about the performance and progress of other users with similar characteristics. The system may do so by comparing the user's performance data from the last treatment session with aggregated data collected by the system for a population of users. The system may then generate a recommended treatment program for the user's next appointment based on the comparison of the user's information and stats with the data for the population of users.

In some embodiments, the aggregated data may include a performance database based on the demographic and medical data about users and their related workout sessions. This performance database will include and accumulate a qualitative measure from the user about their experience (e.g. pain, satisfaction) during the session. In further embodiments, the aggregated data may include and accumulate data from medical personnel (e.g. physical therapists supervising users) as to the outcome of a user's treatment session. This data will also be stored in the performance database.

In some embodiments, the user may not have any prior experience with the assistive devices or training systems (either DAP or non-DAP unweighting). In such cases, the systems described can design a suitable treatment based on the user's information. For example, a user with no prior DAP system experience may wish to use DAP to improve the user's running speed. To design the appropriate DAP system, the treatment management and scheduling system may receive the user's information regarding the desired treatment result. In this example, the user may input into the treatment and scheduling system that she wants to decrease the time needed for her to run a mile. The user may optionally input additional information regarding her location and the time slot for the treatment. The treatment and scheduling system then employs a predictive algorithm, such as the ones described above, to determine the appropriate treatment and DAP system for the user. The predictive algorithm may compare the user's information to that in a database with aggregate data (including performance data) regarding the population of users that have used a DAP system. The algorithm then assesses the treatment parameters employed by other users to determine what treatment would be suitable for the user. The treatment management and scheduling system may then provide one or more suggested treatments to the user and have the user decide on a treatment.

In the case where multiple treatment options are available, the user may first decide on the type of treatment. Once that is selected, the treatment management and scheduling system may then determine which training system, progression of systems or other rehabilitation equipment can provide that treatment regime. For example, if the algorithm determines that users can improve running speed by modifying gait or by running under positive pressure, the system may offer those two treatment options to the user. If strength or flexibility improvement is needed along with use of the DAP system, for example, then scheduling system can recommend treatments involving multiple modes of therapy. If the user picks gait modification as a treatment, the treatment management and scheduling system may then match the user with DAP systems having gait analysis capability. Alternatively, the treatment management and scheduling system may offer the non-DAP unweighting systems to the user and indicate in the listing that the non-DAP system selected can provide gait or an alternative unweighted treatment.

### Example 1

Example 1 provides for an exemplary treatment management and scheduling system that can return a list of nearby facilities with DAP systems based on distance. In addition, the exemplary treatment management and scheduling system provides the user a visual indication of how “available” appointments are at each location, based on time slots, required equipment, etc. In this example, the user selects a facility from the list based on their preference. The treatment management and scheduling system shows the user information about that facility, including a calendar. Hovering over a date on the calendar shows the number of appointments available on that date. The calendar squares are colored coded to indicate days which are relatively open and days which are almost full. Dates without any available appointments are grayed out. The treatment management and scheduling system has a provider interface which allows the provider to specify their regular office hours for each day of the week. The provider can also specify regular blocks of time which are not available for scheduling by the system. Additionally, the provider can specify dates when the office will be closed (e.g. holiday or vacation).

The user selects a specific date from the calendar. The system displays the appointments available for that day. The user selects their desired appointment time. The system displays a form to enter user information (minimally, name, phone number, e-mail address, and credit card information for payment). The user enters the required information. The system re-displays the information provided by the user, in order to confirm its correctness. The user confirms the information. The system uses the payment information to charge the patient. Assuming the payment transaction is successful, the system displays a confirmation message and code. At the same time, the system notifies the facility of the confirmed appointment (possibly by directly interfacing with its scheduling system), and e-mails the confirmation message and code to the user. The e-mail sent to the user will also include a small set of initial questions, such as the reason for the appointment, their doctor's recommendation for weight-bearing, etc. The answers to these questions will be sent to the facility/therapist in advance of the appointment. The questions sent to the user will include questions (e.g. health plan, diagnosis) which enable the system to determine the extent of insurance coverage and obtain pre-authorization for that treatment by the facility.

If a performance database (as described above) is available, the questions for the user will be refined to match the best inputs for use with predictive treatment algorithms. The session parameters recommended by these algorithms will be sent to the therapist for review and approval. If these are approved, the parameters will be transmitted to the DAP system for the scheduled session. In the event that the suggested session parameters require preauthorization with insurance, or other reimbursement related activity, the system can notify the user and/or the therapist. Where the user's medical information is known, the system may notify the user's physician.

Between the time the user schedules the appointment and the appointment date, the system will send appropriately timed reminders (e.g. a week in advance of the appointment, the day before the appointment, an hour before the appointment).

On the day of the scheduled session, the system downloads the proposed workout to the treadmill. If the therapist has not already approved the workout, a reminder is sent to them. The user arrives at the scheduled time and identifies himself to the machine. The system queries them as to their initial state (e.g. level of pain, overall mood). The user performs the workout, with the system recording their performance data. The user rates their workout experience (pain, mood). The therapist rates the workout experience and provides their recommendation for the next session, using an input device connected to the system (touch screen, keyboard/keypad, or even paper which can be scanned).

Using performance data, user rating and therapist inputs, the system creates documentation of the session suitable for inclusion in the patients file (e.g. a PDF file which can be printed or attached electronically). The performance data and user/therapist ratings are added to the performance database, associated with this user. The system sends a reminder to the user to schedule their next appointment, along with suggested date based on data analysis (best appointment interval to maximize recovery based on analysis of aggregate data). The system will also send congratulatory messages to the user as they reach certain treatment milestones. Based on aggregate data, the system will predict and schedule the number of appointments needed by the user.

If the user's sessions are covered by insurance, the system will maintain the information required to generate required reporting. In the event that coverage ends before the expected outcome has been reached, the system will generate the required documentation to petition for additional treatment coverage. The system will also inform the user of their coverage (e.g. number of sessions covered by insurance, number remaining). If insurance information is provided, the system may automatically submit for reimbursement, and may submit supporting data as appropriate.

If the predictive algorithm shows that the number of covered sessions remaining is less than the number of treatments predicted to achieve the desired outcome, the system will generate a reminder to the facility/therapist that re-authorization is required (as generate the required documentation as above). The system may also directly submit requests for additional insurance authorizations if additional sessions are required to achieve treatment objectives and if insurance information has been entered for the patient.

The system can be extended to scheduling other aspects of treatment such as bikes, isometric or isokinetic equipment, diagnostic procedures and traditional rehab or other medical interventions integrated with exercise protocols or influenced by progress in the patient's rehab.

Predicted Results: User successfully schedules and conducts treatment session. The treatment management and scheduling system will record the user's progress and account for the user's treatment session in scheduling future sessions.

### Example 2

Example 2 provides an exemplary treatment management and scheduling system for matching a user with a DAP system. A patient with factors A, B, C, and D (e.g. age, gender, primary diagnosis, secondary diagnosis) has those factors prioritized as follows: C, A, D, B (C being highest priority and B being lowest priority). The patient is scheduling an appointment for the first time and therefore there is no prior data in the system for this patient. Initially, the treatment management and scheduling system will attempt to retrieve information regarding the user from the system's storage database as well from any storage medium in a DAP system that is network connected with the treatment management and scheduling system.

If no information is retrieved, the treatment management and scheduling system will attempt to retrieve information regarding all patients who have factors A-D from all available databases. If there are enough such patients (threshold value), the system moves to the next step. In some cases, the threshold value is based on statistical significance or values determined empirically.

If the threshold value is not satisfied, the system removes the least critical factor (in this example, B) and performs a database retrieval for patients with factors A, C, and D. If this fails to return sufficient results, the system removes factor D from consideration, and retrieves patients with factors A and C. Regardless of the number of factors, the system eventually retrieves sufficient results for the next step, or runs out of factors to consider.

If the system runs out of factors, the system uses a “default” first appointment profile for scheduling (e.g. a 1 hour appointment on any type of DAP treadmill). Note that the system may require a certain amount of pre-existing data, and initially may provide this default appointment to all patients, regardless of the factors they possess. Alternatively, an appointment could be based on a set of rules (created by experts) created for a variety of likely initial conditions, or the system could default to only certain types of DAP systems that serve the broadest population of patients if the user does not enter sufficient information to determine that the user would qualify for a system with more limited capabilities.

If the system does not run out factors, the system retrieves the data about the 1st appointment for each of the matching patients, and proceeds to analyze it. For example, the system may evaluate the data regarding matching patients and determine that patients with the most successful rehabilitation outcomes (or who were most satisfied with their first appointment, or some other factor deemed important) tended to have first appointments that lasted 1 hour. So the system would schedule a 1 hour appointment block for the first appointment.

Similarly, the system may determine that for the patient sample retrieved, a patient's first workout averaged 5 minutes at 2 mph with a 0% incline and 50% body weight. The calculation of that average could include a weighting to again reflect the outcome of the patient's treatment. The system would deliver a workout with those specifications to the DAP treadmill that is ultimately scheduled for this appointment.

For scheduling, the system is configured to only schedule a machine which can provide the specified workout (for example, a workout requiring incline requires a treadmill which is capable of incline. DAP treadmills vary in terms of the maximum speed they can deliver, whether they can operate in reverse, whether they can incline (and how much), the amount of “unweighting” they can provide, and the degree of sensor and patient data collection and analysis provided. As such, the treatment management and scheduling system takes into account the variability of DAP systems. For example, a patient with gait anomalies or with a medical condition that is likely to result in gait anomalies such as orthopedic surgery of one lower limb would be best served in a machine with gait diagnostic capabilities that could facilitate faster improvement and better tracking of improvement over successive sessions. Directing such a patient to a DAP system capable of gait diagnostics may be important particularly for the initial visit to get an accurate baseline assessment and develop appropriate treatment recommendations.

Once the system has information such as the length of appointment and the type of DAP treadmill, and possibly other requirements (e.g. all stroke patients of a certain severity require a lift access or multiple personnel to assist the patient when entering/exiting the DAP treadmill), the system can locate DAP treadmills matching the equipment (and possibly other) requirements. If no matches are found, the system will log the fact and request the user to contact customer support.

The system can also calculate (based on facility data in the system) which facilities within a specified distance of the patient (e.g. 120 miles) have appointment times matching the appointment length requirement. It counts the number of one hour appointments which are available over a specified time period (e.g. 7 days). The system can also present the facilities in order of distance with an indicator (e.g. green, yellow, red) showing the number of appointments matching the patient's needs that are available at each facility.

If no matches are found, the system logs the fact and requests the user to contact customer support.

Predicted Results: User successfully schedules and conducts treatment session. The user selects a facility from the list and an appointment time from the associated calendar for that facility.

### Example 3

Example 3 provides an exemplary treatment management and scheduling system that can be used to apportion revenue between the owner of the system and the facility with the scheduled DAP system. In this example, a user comes to the website and books an appointment for a DAP system at a facility. The user pays $20 by credit card for their first appointment. The system issues that user a unique ID to use the machine (which must be provided by the user in order to use the machine).

The user keeps the appointment, entering her ID into the treadmill console. The system (via the treadmill's wireless connection) receives notification that the user has kept the appointment. When that notification is received, the system generates a $10 (50% of $20) ACH transfer to the facility (the facility has previously supplied its bank routing/account number). Alternatively, the system can generate a notification to the treatment management and scheduling system's owner to provide the facility with $10.

When the user schedules her next appointment with the facility, the system is either notified by the facility or is aware of the appointment via integration between the treatment management and scheduling system and the DAP systems.

After the user keeps the appointment, entering her ID into the treadmill console. The system receives notification, and generates a PDF invoice for $5 (40% of $20) which is e-mailed to the facility accounting department (with a copy to the treatment management and scheduling system's owner's accounting department). In some cases, the treatment management and scheduling system may be integrated with the accounting systems.

As a fraud prevention feature, the system will send a query to the facility where a user's ID is entered on the DAP treadmill for an ID where (a) a revenue split applies, and (b) no appointment is scheduled is logged.

After the fifth appointment is kept by the user, the system no longer pays/invoices the facility.

Predicted Results: The revenue will be shared according the formula f(n,r) described above where, the function returns 0 for n>5, and (r*(60%−(10%*n))) for 1≤n≤5.

### Example 4

This example provides an exemplary treatment management and scheduling system that allows a therapist to create or modify pre-programmed workout sessions and attach these to an appointment scheduled by the user, overriding any system-generated workout session. The system, based on aggregate data collected from users with similar conditions and the past history of the user, generates a suggested DAP treadmill workout as a series of segments (e.g. duration, speed, incline, percentage of body weight) for the user as part of scheduling their next appointment. The system, prior to the appointment (generally on the prior business day) reminds the supervising therapist or trainer of the appointment and send them a link to a secure web page. The therapist/trainer logs in to the secure web page, which displays the workout segments planned for the user. The therapist/trainer adjusts the workout by editing individual segments, either changing one or more of the segment parameters (duration, speed, incline, body weight), or removing the segment altogether. Additionally, the therapist/trainer can add a new segment before or after any existing segment, again by specifying its parameters. When the therapist/trainer is satisfied with the workout, they save the edited version.

Alternatively, the therapist/trainer can delete the pre-programmed workout and create one from scratch, a segment at a time, specifying the parameters for each segment. The therapist/trainer can save the new version when complete. The system attaches the newly-saved workout to the user's appointment. Instead of performing these activities on a web page, the therapist/trainer might perform them on the DAP device itself, possibly by means of a control panel or associated tablet device.

At the appointed time, the workout is downloaded into the machine for use when the user enters his ID code.

Predicted Results: User successfully schedules and conducts treatment session after physical therapists adjusts treatment program.

### Example 5

This example is of an exemplary treatment management and scheduling system that allows a sequence or series of appointments to be scheduled, based on either a number (e.g. 10 appointments) or a desired outcome (e.g., walking at 3% incline at 2 mph at 95% of body weight). The treatment management and scheduling system receives a user's information such as age, gender, and initial diagnosis. The system then evaluates all patients in the aggregated data database that match the current patient. To do so, the system uses a list of prioritized criteria (as detailed above) and a predictive algorithm to predict both the number of appointments which the patient will make before ending treatment and the exit condition for ending treatment (in terms of the patient's treatment/workout profile). Based on that prediction, the system can schedule an optimal sequence of appointments, both in terms of the time between appointments and the DAP treatment/workout to be conducted in each appointment.

In cases where the user stops treatment before the goal is reached, the system can query the patient via e-mail as to the reason. In cases where the patient felt that they were “back to normal”, this information can be used to improve the prediction algorithm. The system can respond to other reasons (such as change in insurance coverage, a change of location, etc.) appropriately to re-engage the patient with treatment.

At the predicted end of treatment (as dictated by desired outcome) the system will query the user and, if applicable, the physical therapist to see if the outcome has been reached and use this information to improve future predictions. When the treatment is actually completed (as indicated by user or therapist), this information will be added to improve further predictions. Additionally, if the user schedules ten appointments and the normal course of treatment is known to be more or less than that number, the system can inform the patient and possibly involve outside resources (insurance, therapist). As the user proceeds through the appointments, the system will provide feedback as to how he is progressing against the predicted pattern.

Predicted Result: User will schedule a series or sequence of sessions for treatment based on a recommended treatment generated by the treatment management and scheduling system. The treatment management and scheduling system will employ a predictive algorithm to match the patient with an appropriate treatment plan. The predictive algorithm will take into account the user's information and compare the user with a population of users whose information is available in an aggregated information database. Based on prioritized matching, the predictive algorithm will provide the user with an appropriate treatment.

The above described techniques for determining, monitoring, refining and tracking a user's training, rehabilitation or process may be applied to a wide variety of systems and using a wide array of devices to help the user. Exemplary devices and systems that may be incorporated into the techniques described herein include: U.S. Provisional application titled “SYSTEMS AND METHOD FOR MANAGEMENT AND SCHEDULING OF DIFFERENTIAL AIR PRESSURE TREATMENT,” Application No. 61/654,410, filed Jun. 1, 2012, attorney no. 11889-706.100; U.S. Provisional application titled “METHOD OF GAIT EVALUATION AND TRAINING WITH DIFFERENTIAL PRESSURE SYSTEM,” Application No. 61/651,415, filed May 24, 2012, attorney no. 11889-705.100; U.S. Provisional application titled “METHOD OF GAIT EVALUATION AND TRAINING WITH DIFFERENTIAL PRESSURE SYSTEM”, Application No. 61/785,317, filed Mar. 14, 2013, attorney no. 11889-705.101; U.S. Provisional application titled “UNWEIGHTING CAGE,” Application No. 61/784,387, filed Mar. 14, 2013, attorney no. 11889-708.100; U.S. Provisional application titled “CANTILEVERED UNWEIGHTING SYSTEM,” Application No. 61/784,510, filed Mar. 14, 2013, attorney no. 11889-713.100; U.S. Provisional application titled “UNWEIGHTING GARMENTS,” Application No. 61/773,048, filed Mar. 5, 2013, attorney no. 11889-712.100; U.S. Provisional application titled “UNWEIGHTING GARMENTS,” Application No. 61/784,664, filed Mar. 14, 2013, attorney no. 11889-712.101; U.S. Provisional application titled “CURVED ARCH UNWEIGHTING SYSTEMS,” Application No. 61/772,964, filed Mar. 5, 2013, attorney no. 11889-709.100; U.S. Provisional application titled “UNWEIGHTING ARCH SYSTEMS,” Application No. 61/773,019, filed Mar. 5, 2013, attorney no. 11889-710.100; and U.S. Provisional application titled “MONOCOLUMN UNWEIGHTING SYSTEMS,” Application No. 61/773,037, filed Mar. 5, 2013, attorney no. 11889-711.100. Each of the above described patent application is incorporated herein by reference for all purposes.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

### APPENDIX

DAP systems with treadmills are typically sold to physical therapists, skilled nursing facilities, hospitals, rehabilitation clinics, health clubs, and athletic organizations. Organizations which make their DAP treadmills available to the public would like to see high levels of utilization to maximize their return on investment. There is a need to attain full utilization of each machine, which will increase the chances of selling additional machines to meet demand. To that end, it is important to know the status of each machine in real time.

Machine owners want to be paid for the use of their machine. A treatment management and scheduling system can help generate usage (i.e. revenue) for an owner. That revenue may be shared with owner of the treatment management and scheduling system.

Finally, like airline seats and hotel rooms, time on a DAP system becomes valueless with the passage of time. This opens the potential for differential pricing to drive demand.

A system of scheduling which improves the return on investment for machine owners by scheduling machines to maximize utilization and revenue is likely to be widely adopted, regardless of the method currently in use.

What is claimed is:

1. A method of differential air pressure (DAP) system treatment management, comprising
providing a user's information, the information comprising at least two of the following characteristics: age, weight, gender, location, desired result, current medical condition, height, lift access requirements, therapist access requirements, therapy history, past workout information, and user type, wherein user type comprises at least one of an athlete, a casual user, a rehabilitation user, and a chronic user;
analyzing, using a processor, the user's information based, at least in part, on aggregate information in a database comprising other users' characteristics and associated past workout session data including duration, speed, incline, and unweighting level used during workouts; and
generating, using a processor, a suggested workout routine including duration, speed, incline, and unweighting level to be used during a workout based on the comparing of the user's information to the other users' information.
